(19) United States
(12) Plant Patent Application Publication
(10) Pub. No.: US 2005/0241037 P1
(43) Pub. Date: Oct. 27, 2005

(54) Nectarine tree named "NECTAPINK"

(75) Inventors: Laurence Maillard, Elne (FR); Arsene Maillard, Elne (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: S.A.R.L. AGRO SELECTION FRUITS, Elne (FR)

(21) Appl. No.: 11/115,248

(22) Filed: Apr. 27, 2005

(30) Foreign Application Priority Data
Apr. 27, 2004 (FR) 2004-0741

(51) Int. Cl. A01H 5/00

(52) U.S. Cl. PLT/190

(57) ABSTRACT

A new and distinct variety of nectarine tree, denominated “NECTAPINK”, has a very long fruit lifetime, and bearing fruit having a sweet sub-acid yellow flesh of high eating quality and an attractive, very high percentage red skin. The tree is of medium size and is vigorous. Moreover fruit can be consumed crunchy or at maturity.

This application claims priority of Community plant variety right No. 2004-0741 filed on Apr. 27, 2005 which is hereby incorporated by reference in its entirety.
Botanical classification: Prunus persica sylvestris—Yellow Nectarine.

### BACKGROUND OF THE NEW VARIETY

The present invention relates to a new and distinct variety of [Prunus persica sylvestris—Yellow] nectarine tree. This new tree, named “NECTAPINK”, produces very long lifetime, good eating quality, semi-freestone flesh fruit for fresh market in July in the Pyràenèes Orientales (66), France. Contrast is made to “NECTAROSS” and “ZAITABO” nectarines, standard varieties, for reliable description. “NECTAPINK” is a promising candidate for commercial success in that it has an early flowering and a very long lifetime and durability of its fruit.

### ORIGIN OF THE VARIETY

“NECTAPINK” nectarine tree (genotype) originated in a cultivated area of the south of France, in the Pyràenèes Orientales (66) where it was tested. The female parent was “ZAITABO” yellow nectarine (BIG TOP®) and the male parent was a non-diffused variety obtained from seedbed MAILLARBELLE white nectarine. “NECTAPINK” was provisionally designated and tested as 01.08.60.01 NJ and is registered at the Official Catalogue of the Agriculture Ministry of the French Republic Dec. 17, 2003 under number 1014086. It was asexually obtained by hybridizing and is propagated by grafting. It has been determined to have unique tree and fruit characteristics making it worthy for commercial fresh fruit production. There are no known effects of this standard rootstock on this scion cultivar. Asexually propagated plants remained true to the original tree and all characteristics of the tree and the fruit were transmitted. The plant was reproduced asexually on the property of S.A.R.L. Agro Selection Fruits,

Les Règalines, Route d'Alenya, La Parde de Mousseillous, 66200 ELNE, Pyràenèes Orientales, France.

### SUMMARY OF THE VARIETY

The new and distinct variety of nectarine tree blooms with “NECTAPINK” nectarine in early March at Perpignan in the Pyràenèes Orientales, France. More particularly, it blooms from 4 to 5 days before “NECTAROSS” and “ZAITABO”, between 1^{st} and 16^{th} of March.

The first fruit ripens at the end of July and early august, at the same time as “NECTAROSS”, nearly one month after “ZAITABO”. More particularly, it approximately ripens between July 28 and August 8.

### DESCRIPTION OF THE DRAWINGS

In the accompanying drawing, which are as nearly true as it is reasonably possible to make in a color illustration of this type:

FIG. 1 is a color photograph which shows a typical specimen of the leaf, stem and flower of the new variety.

FIG. 2 is a color photograph which shows a typical specimen of the fruit of the new variety.

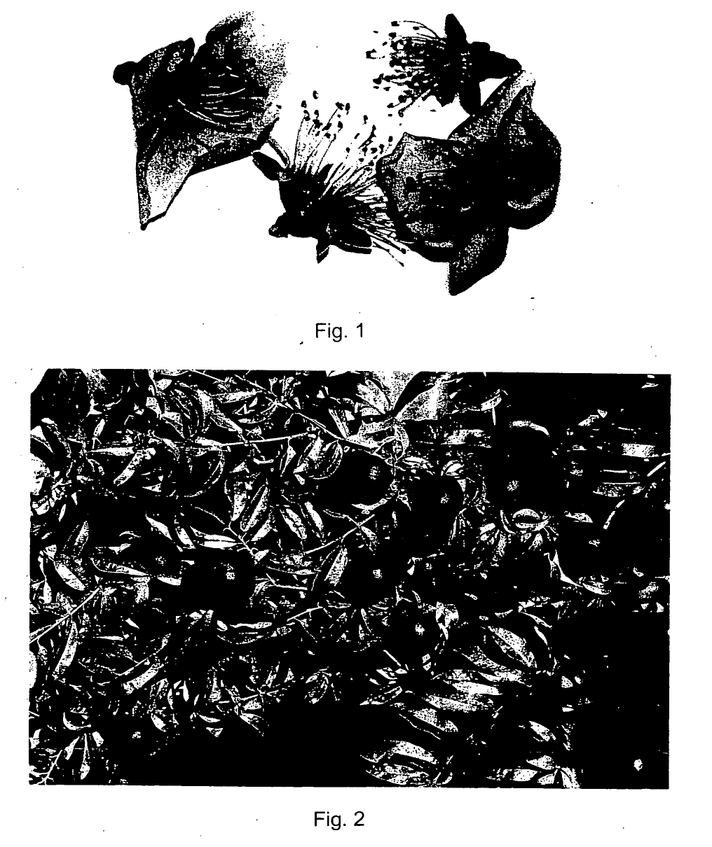

### DETAILED BOTANICAL DESCRIPTION

The tree, flowers, and fruit may vary in slight detail due to variations in soil type, cultural practices, and climatic condition. The potential for commercial production of fresh fruit by “NECTAPINK” is high, due to its long fruit lifetime.

Trees are vigorous and medium stature half-standing in a semi-spread out aspect. The flowering shoot is present excluding brindilles side away from sun. Flowering begins early in springtime. The type of flower are showy and rosaceous with relative large and medium petal size. Petals are pale pink. Nectarine petioles are present and reniform. Time of maturity for consumption is medium. The fruit flesh is yellow with a lightly red pigmentation around the stone and its skin is thick very red with a yellow background. The stone is medium size and the flesh is very adherent. Fruit taste is sub-acid and sweet.

Compared to NECTAROSS variety, NECTAPINK has the same time of maturity although the red coloration is higher, 90 to 100% instead of 60 to 70%, and the taste is sweeter instead of being semi-acid. Lifetime of fruit is very long instead of being short.

Moreover fruit can be consumed crunchy or at maturity.

Tree and fruit are disease resistant to chlorotic leaf spot, necrotic ring spot, prune dwarf, sharka (plum pox), nepovirus, peach latent mosaic and peach yellows. They are not sensible to cryptogamic disease.

### DETAILED DESCRIPTION

Referring more specifically to the pomological details of this new and distinct variety of nectarine tree, the following has been observed during the fruiting season under the ecological conditions prevailing at Elne which are located in the Pyràenèes Orientales, France.

### TREE

Size:
   Generally.—Considered medium and large as compared to other common commercial nectarine cultivars ripening in the middle season of maturity.
Vigor: Considered vigorous.
Productivity: Very Productive. Fruit set is spaced by thinning to develop the remaining fruit into the desired market sized fruit. The number of the fruit set varies with the prevailing climatic conditions, and cultural practices employed during the bloom period, and is therefore not distinctive of the present variety.
Bearer: Regular. Fruit set has been heavy during the years of observation and thinning was necessary during the past 3 years.
Form: Upright semi-standing, and pruned to a vase semi-spread out shape.
Density: Considered medium dense.
Hardiness: The present tree was grown and evaluated in France. The variety appears to be hardy under typical central Pyràenèes Orientales climatic conditions.

### TRUNK

Diameter: Approximately 11.2 cm in diameter when measured at a distance of approximately 15.24 cm above the soil level. The measurement was taken at the end of the third growing season.
Bark texture: Considered moderately rough, with numerous folds of papery scarfskin being present.
Lenticels: Numerous flat, oval lenticels are present.
Lenticel color: Considered an orange brown.
Bark coloration: Variable, but it is generally considered to be a medium grey-brown.

### BRANCHES

Size: Considered medium for the variety.
Diameter: Average as compared to other nectarine varieties.
Surface texture: Average, and appearing furrowed on wood which is several years old.
Crotch angles: Primary branches are considered variable. This particular characteristic is not considered distinctive of the variety, however.
Current season shoots:
   Surface texture.—Substantially glabrous.
Internode length: Considered average.
Color of mature branches: Medium brown.
Current seasons shoots:
   Color.—Light green. The color of new shoot tips is considered a bright and shiny green.

### LEAVES

Size: Considered medium to large for the species. Leaf measurements have been taken from vigorous, upright, current-season growth at approximately mid-shoot.
Leaf length: Considered average.
Leaf width: Considered average.
Leaf base shape: Slightly oblique relative to the leaf longitudinal axis.
Leaf form: Lancelolate. Considered to be relatively narrow for the species.
Leaf tip form: Reniform.
Leaf color:
   Upper leaf surface.—green.
Leaf texture: Glabrous.
Leaf color: green.
   Lower surface.—Medium green.
Leaf venation: Pinnately veined.
Mid-vein:
   Color.—Light yellow green.
Leaf margins: Slightly undulating.
Form: Considered crenate.
Uniformity: Considered generally uniform.
Leaf petioles:
   Size.—Considered medium.
   Length.—About 8.0 to about 10.0 mm.
   Diameter.—About 1.0 to about 2.0 mm.
   Color.—Pale green.
Leaf glands:
   Size.—Considered medium.
   Number.—Generally one gland per margin side. Occasionally two glands per margin side.
   Type.—Reniform.
   Color.—Considered a pale orange.
Leaf stipules:
   Size.—Medium large for the variety.
   Number.—Typically 2 per leaf bud and up to 4 per shoot tip.
   Form.—Lanceolate in form and having a serrated margin.
   Color.—Green, when young, but graduating to a brown color, with advancing senescence. The stipules are considered to be early deciduous.

### FLOWERS

Flower buds:
   Generally.—The floral buds, depending upon the stage of development, are approximately 7.0 millimeters wide; about 11.0 millimeters long; conic in form; and slightly appressed relative to the bearing shoot.
Flower buds:
   Color.—This characteristic is dependent upon the proximity to bloom. The bud scales are deep purple. The buds are considered hardy under typical central Pyràenèes Orientales climatic conditions.
Hardiness: No winter injury has been noted during the last several years of evaluation in the central Pyràenèes Orientales. The current variety has not been intentionally subjected to drought or heat stress, and therefore this information is not available.
Date of bloom: early March.
Blooming time: Considered early-season in relative comparison to other commercial nectarine cultivars grown in the Pyràenèes Orientales, France. The date of full bloom is observed on March. The date of bloom varies slightly with climatic conditions and cultural practices.
Duration of bloom: Approximately 16 days. This characteristic varies slightly with the prevailing climatic conditions.
Flower type: The variety is considered to have a showy type flower.
Flower size: Flower diameter at full bloom is considered average.
Bloom quantity: Considered abundant.
Flower bud frequency: Normally 1 to 2 flower buds appear per node.
Petal size:
   Generally.—Considered between medium and large for the species.
Length: Considered average.
Width: Considered average.
Petal form: Slightly ovoid.
Petal count: Nearly always 5.
Petal texture: Glabrous.
Petal color: Pink and light pink.
Fragrance: Slight.
Petal claw:
   Form.—The claw is considered generally ovoid and has a medium size when compared to other varieties.
   Length.—Considered average.
   Width.—Considered average.
Petal margins: Generally considered variable, from nearly smooth to slightly ruffled, to moderately undulate.
Petal apex:
   Generally.—The petal apices are generally entire at the tip.
Flower pedicel:
   Length.—Considered medium-long, and having an average length.
   Diameter.—Considered average.
   Color.—A medium brown.
Floral nectaries:
   Color.—A dull orange.
Calyx:
   Surface texture.—Generally glabrous.
   Color.—Purple.
Sepals:
   Surface texture.—The surface has a short, fine pubescent texture.
   Size.—Average, and ovate in form.
   Color.—A deep purple.
Anthers:
   Generally.—Average in length.
   Color.—Red to reddish-orange dorsally.
Pollen production: Pollen is abundant, and has a yellow color. The present variety is considered self fruitful (self-pollinating).
Filaments:
   Size.—Length is variable.
Color: Considered light pink.
Pistil:
   Number.—Usually 1, rarely 2.
   Generally.—Average in size.
   Length.—Considered average including the ovary.
   Color.—Considered a very pale green.
   Surface texture.—The variety has a long glabrous pistil.

### FRUIT

Maturity when described: Firm ripe condition (shipping ripe).
Date of picking: July and August. The date of harvest varies slightly with the prevailing climatic conditions.
Size:
   Generally.—Considered large, and uniform.
Average cheek diameter: Approximately 70.0 to about 80.0 millimeters.
Average axial diameter: Approximately 60.0 to about 75.0 millimeters.
Typical weight: Approximately from 200.0 to 300.0 grams. This characteristic is high dependent upon the prevailing cultural practices, and therefore is not particularly distinctive of the variety.
Fruit form:
   Generally.—Considerably oblate. The fruit is generally uniform in symmetry.
Fruit suture: Very shallow and smooth, extending from the base to the apex. No apparent callousing or stitching exists along the suture line.
Suture:
   Color.—This has a yellow background color.
Ventral surface:
   Form.—Only slightly indented.
Apex: Rounded.
Base: Generally retuse.
Stem cavity: Generally elongated in the suture plane.
Fruit skin:
   Thickness.—Considered medium in thickness, and tenacious to the flesh.
   Texture.—Glabrous.
   Taste.—Non-astringent.
   Tendency to crack.—None observed.
Color:
   Blush color.—This blush color is generally red. The blush covers approximately 90% -100% of the fruit skin surface. The percentage of the blush on the fruit skin surface can vary, and is generally dependant upon the prevailing conditions under which the fruit was grown.
   Ground color.—Yellow orange.
Fruit stem: Medium in length.
Diameter: Considered average.
Color: Pale yellow-green.
Flesh:
   Ripens.—Evenly.
   Texture.—Firm, juicy and dense. The flesh is considered non-melting.
   Fibers.—Few, small, and tender ones are typically found.
   Aroma.—Very slight.
   Eating quality.—Considered very good.
   Flavor.—Considered sweet and mildly acidic. The flavor is considered both pleasant and balanced.
   Juice.—Moderate.
   Brix.—About 14.0 degrees. This characteristic varies slightly with the number of fruit per tree; prevailing cultural practices; and the surrounding climatic conditions.
   Flesh color.—Pale yellow-orange.

### STONE

Type: Clingstone.
Size: Considered medium for the variety. The stone size varies significantly depending upon the tree vigor, crop load and prevailing growing conditions.
Length: Average.
Width: Average.
Diameter: Average.
Form: Ovoid.
Base: The stone is usually rounded to slightly oblique relative to the ventral side.
Apex:
   Shape.—The stone apex has a small prominent tip.
Stone surface:
   Surface texture.—The pit is irregularly furrowed toward the apical end. Pitting is generally more present and noted on the lateral sides and toward the base.
   Ridges.—The surface texture is generally characterized by more prominent ridges along the ventral edges and is more prominent at the apical tip.
Ventral edge:
   Width.—Considered medium.
Dorsal edge:
   Shape.—Full, lightly grooved, and having a reasonably smooth margin.
Stone color: The color of the dry stone is generally considered a reddish brown.
Tendency to split: Splitting has rarely been noted.
Kernel:
   Size.—The kernel is considered medium-large.
   Form.—Considered ovoid.
   Pellicle.—Pubescent.
   Color.—Greyed orange.
Use: The subject variety “NECTAPINK” is considered to be a nectarine tree of the middle season of maturity, and which produces fruit that are considered firm, attractively colored, and which are useful for both local and very long distance shipping.
Keeping quality: Excellent. Fruit stayed one week on tree before harvest and then, has stored well up to 3 weeks after harvest at 1.0 degree Celsius.
Shipping quality: Considered good. The fruit of the new nectarine variety showed minimal bruising of the flesh or skin damage after being subjected to normal harvesting and packing procedures.
Resistance to insects and disease: No particular susceptibilities were noted. The present variety has not been tested to expose or detect any susceptibilities or resistances to any known plant and/or fruit diseases.

Although the new variety of nectarine tree possesses the described characteristics when grown under the ecological conditions prevailing near ELNE, Pyràenèes Orientales, France, it should be understood that variations of the usual magnitude and characteristics incident to changes in growing conditions, fertilization, pruning, pest control and horticultural management are to be expected.

1. A new and distinct nectarine tree variety as illustrated and described, characterized by a very long fruit lifetime, and bearing fruit having a sweet sub-acid yellow flesh of high eating quality and an attractive, very high percentage red skin.
